(19) United States
(12) Patent Application Publication
(10) Pub. No.: US 2007/0176118 A1
(43) Pub. Date: Aug. 2, 2007

(54) Apparatus and method for sterilizing a spout assembly of a container

(75) Inventor: Lars Aksel Thingelstad, Drammen (NO)

Correspondence Address: CALFEE HALTER & GRISWOLD, LLP, 800 SUPERIOR AVENUE, SUITE 1400, CLEVELAND, OH, 44114, US

(73) Assignee: ELOPAK SYSTEMS AG, GLATTBRUG SWITZERLAND (CH)

(21) Appl. No.: 11/628,950

(22) PCT Filed: Jun. 8, 2005
(86) PCT No.: PCT/GB05/02254

§ 371 Date: Apr. 9, 2007

(30) Foreign Application Priority Data
Jun. 8, 2004 (GB) 0412695.9

(51) Int. Cl. A61L 2/08

(52) U.S. Cl. 250/455.11; 422/22

(57) ABSTRACT

A system for subjecting articles and containers to microbiocidal radiation and for attaching the articles to the containers comprises a source (6) of microbiocidal radiation, a first conveying device (26) for advancing pour spout fitments (21) along a first path, a second conveying device (2) for advancing containers (4) along a second path substantially parallel to the first path, the source (6) being effective along both the first and the second paths, and a fitment applicator (27) arranged to attach to the containers (4) fitments (21) which have been subjected to the radiation.

This invention relates to a method and apparatus for subjecting articles and containers to microbiocidal radiation and for attaching the articles to the containers.

It is known, in a form-fill-seal machine for liquid packaging cartons, to attach pour spout fitments to the bottom-sealed, open-topped cartons before filling the cartons with product. The fitments may be inserted into holes through top closure obturating panels of the respective cartons, with pour spout flanges of the fitments being sealed to inside surfaces of the respective panels and with screw caps of the fitments being situated externally of the panels. In the case of fitments having their hollow interiors communicating with the interiors of the as yet unfilled cartons, it is difficult to sterilize, and even to render ultraclean, the interiors of the fitments by UV sources disposed above the cartons.

Examples of disclosures in which the fitments are in place on the containers before disinfecting or sterilizing are JP-A-05-132,043; JP-A-2003-072,717; U.S. Pat. Nos. 5,129,212; 6,066,081 and 6,094,887.

Examples of disclosures in which the fitments are disinfected or sterilized separately from the containers are EP-A-1291162 [in which the fitments are partly sterilized by ultraviolet (UV) radiation before being applied to carton sleeves which are subsequently sterilized by gaseous hydrogen peroxide (H₂0₂) before bottom sealing and filling] and JP-A-2003-237742 [in which it seems that pouches are externally sterilized by gaseous H₂0₂ whilst their spouts are in sealed conditions, then unsealed and filled, and thereafter sealed by fitments which have meanwhile been sterilized by gaseous H₂0₂].

According to one aspect of the present invention, there is provided a method comprising subjecting articles to microbiocidal radiation while the articles advance along a first path, attaching the thus subjected articles to containers, and subjecting the containers to microbiocidal radiation while the containers advance along a second path substantially parallel to the first path.

According to another aspect of the present invention, there is provided apparatus comprising a source of microbiocidal radiation, a first conveying device for advancing articles along a first path, a second conveying device for advancing containers along a second path substantially parallel to the first path, said source being effective along the first and second paths, and an article applicator arranged to attach to the containers articles which have been subjected to said radiation.

According to a third aspect of the present invention, there is provided apparatus comprising an elongate, first conveying device for conveying articles along a first path, a second conveying device for conveying containers along a second path substantially parallel to said first path, and an elongate source of microbiocidal radiation extending substantially parallelly to the first and second paths for subjecting said articles and said containers to said radiation.

Owing to the invention, it is possible to employ the same source of microbiocidal radiation for both the containers and the articles.

The articles may be applied to the containers after both have been subjected to the microbiocidal radiation, in which case they are advantageously advanced in the same direction along the respective first and second paths. Alternatively, the articles may be applied to the containers after the articles have been subjected to the microbiocidal radiation but before the containers are subjected to the microbiocidal radiation, in which case they are advantageously advanced in opposite directions along the respective first and second paths.

The containers may be cartons and the articles may be pour spout fitments or lids.

In order that the invention may be clearly and completely disclosed, reference will now be made, by way of example, to the accompanying drawings, in which:

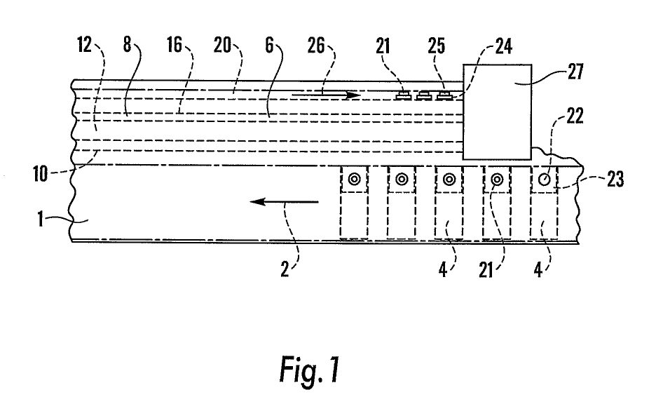

FIG. 1 is a fragmentary side elevation of a form-fill-seal liquid packaging machine; and

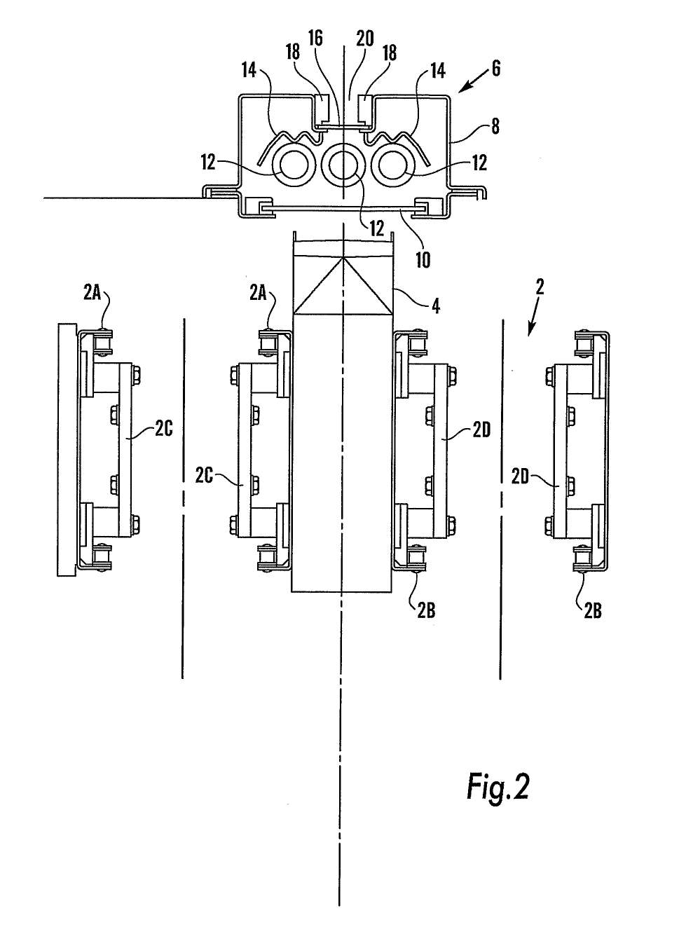

FIG. 2 shows diagrammatically a fragmentary vertical section through the machine.

Referring to the drawings, the machine includes an elongate, horizontal, aseptic chamber 1 through which extends, substantially parallelly to the walls of the chamber, a horizontal conveying device 2 comprised of two horizontal endless chains 2A and 2B and horizontal guide rails 2C and 2D for the respective chains. The inner runs of the two chains 2A and 2B bound pockets for receiving respective bottom-sealed, open-topped cartons 4 to be filled with liquid, for example milk or fruit juice. The cartons may be of laminate material, in particular with a paperboard substrate and interior and exterior coatings of a moisture barrier, such as LDPE (low density polyethylene), possibly with the interposition of an oxygen barrier layer, such as aluminium foil or EVOH (ethylene vinyl alcohol).

Extending parallelly to the conveying device 2 is an elongate radiating device 6 which emits UV (ultraviolet) radiation of microbiocidal wavelength, in particular UV-C. The device 6 includes a housing 8 which in cross-section is mainly of material which is substantially opaque to such UV radiation, but has most of its bottom wall formed of a strip 10 of material, such as quartz glass, which is not opaque to such UV and therefore enables a group of three, UV-emitting tubes 12 parallel to the device 2 to irradiate the cartons 4 below them, in particular their interiors. Elongate reflectors 14 above the two outer tubes 12 reflect UV downwardly towards the cartons 4. Above the middle tube 12, the top wall of the housing 8 is centrally provided with a strip 16 of material, such as quartz glass, which is not opaque to such UV radiation but which transmits UV emitted upwardly from the middle tube 12 to two profiled strips 18 also of material, such as quartz glass, which is not opaque to such UV radiation. The strips 16 and 18 define among them a channel 20 shaped to guide therealong pour spout fitments 21 which are to be mounted in holes 22 through respective top obturating panels 23 of the respective cartons 4. Each fitment 21 may comprise a tubular pour spout which is externally threaded and has an annular, outwardly-projecting flange at its lower end which is to be sealed to the inner coating of the container, as well as a screw cap 25 screwed onto the pour spout. As the fitments are advanced along the channel 20 by a second conveying device 26 the UV radiation transmitted by the walls 16 and 18 of the channel 20 renders sterile or at least ultraclean the inside surfaces of each fitment 21, again possibly with the prior application of H₂0₂.

The channel 20 may be about 800 mm. long. As to the length of time of exposure of each fitment to the UV, this depends upon the size of the pour spout flange and the speed of the machine, because the length of the UV source is predetermined, whilst the number of spouts required is determined by the machine speed, i.e. the number of cartons per hour. The larger the external diameter of the pour spout flange, the lesser the number of fitments which can be present over the UV source, whilst the higher the machine speed, the faster the fitments have to be advanced past the UV source. Owing to the guiding of the fitment 21 by, in particular, quartz glass, and owing to the size and profile of the channel 20 no parts of the interiors of the fitments are shadowed. It would alternatively be possible to arrange for both the exteriors and the interiors of the fitments to be sterilized in a similar manner to that described above, but that would not normally be performed, since it is the interior of the fitment which comes into contact with the filled product.

As they leave the channel 20, the fitments 21 are transferred, in an at least ultraclean environment, to a fitment applicator 27 which attaches the fitments to respective cartons 4 which have not yet been rendered at least ultraclean. These cartons to which the fitments have been applied are then advanced under the three tubes 12 by the conveying device 2 in the direction opposite to the direction of advance of the fitments. As the containers 4 are advanced by the device 2, they are made sterile or at least ultra-clean by the UV radiation from the source 6, possibly with the prior application of H₂0₂ (hydrogen peroxide). Subsequently, the cartons are filled with product and then top-sealed.

1.-18. (canceled)

19. A method comprising subjecting articles to microbiocidal radiation while the articles advance along a first path, attaching the thus subjected articles to containers, and subjecting the containers to microbiocidal radiation while the containers advance along a second path substantially parallel to the first path.

20. A method according to claim 19, wherein said second path is at least partly co-extensive with said first path.

21. A method according to claim 19, wherein the microbiocidal radiation to which said articles are subjected and the microbiocidal radiation to which said containers are subjected radiates from a source common to both said articles and said containers.

22. A method according to claim 19, wherein said fitments are applied to said containers after both have been subjected to the microbiocidal radiation.

23. A method according to claim 22, wherein said articles and said containers are advanced in the same direction along the respective first and second paths.

24. A method according to claim 19, wherein said articles are applied to the containers after the articles have been submitted to the microbiocidal radiation but before the containers are subjected to the microbiocidal radiation.

25. A method according to claim 24, wherein the articles and the containers are advanced in opposite directions along the respective first and second paths.

26. Apparatus comprising a source of microbiocidal radiation, a first conveying device for advancing articles along a first path, a second conveying device for advancing containers along a second path substantially parallel to the first path, said source being effective along the first and second paths, and an article applicator arranged to attach to the containers articles which have been subjected to said radiation.

27. Apparatus according to claim 26, wherein the first and second conveying devices are arranged to advance in the same direction as each other and said applicator is arranged to apply said articles to said containers after both have been subjected to the microbiocidal radiation.

28. Apparatus according to claim 26, wherein the first and second conveying devices are arranged to advance in respective opposite directions and said applicator is arranged to apply said articles to said containers after said articles have been subjected to the microbiocidal radiation but before said containers are subjected to the microbiocidal radiation.

29. Apparatus according to claim 26, wherein said source is an elongate source extending substantially parallelly to the first and second paths.

30. Apparatus according to claim 26, wherein said source comprises a device which includes a radiation emitter and a housing which in cross-section is mainly of material which is substantially opaque to the radiation, but at the bottom thereof, which is above said first path, is not opaque to the radiation and thereby enables said emitter, which is above said bottom, to irradiate said containers therebelow, said device also including reflectors which reflect radiation from said emitter downwardly towards said containers, and, between said reflectors, a strip of material which is below said second path and which is not opaque to the radiation but which transmits the radiation upwardly from said emitter to said second path.

31. Apparatus according to claim 30, wherein said device further comprises first and second profiled strips also of material which is not opaque to the radiation, said strip and said profiled strips defining among them a channel shaped to guide said articles therealong.

32. Apparatus comprising an elongate, first conveying device for conveying articles along a first path, a second conveying device for conveying containers along a second path substantially parallel to said first path, and an elongate source of microbiocidal radiation extending substantially parallelly to the first and second paths for subjecting said articles and said containers to said radiation.

33. Apparatus according to claim 32, wherein the first and second conveying devices are arranged to advance in the same direction as each other and said applicator is arranged to apply said articles to said containers after both have been subjected to the microbiocidal radiation.

34. Apparatus according to claim 32, wherein the first and second conveying devices are arranged to advance in respective opposite directions and said applicator is arranged to apply said articles to said containers after said articles have been subjected to the microbiocidal radiation but before said containers are subjected to the microbiocidal radiation.

35. Apparatus according to claim 32, wherein said source comprises a device which includes a radiation emitter and a housing which in cross-section is mainly of material which is substantially opaque to the radiation, but at the bottom thereof, which is above said first path, is not opaque to the radiation and thereby enables said emitter, which is above said bottom, to irradiate said containers therebelow, said device also including reflectors which reflect radiation from said emitter downwardly towards said containers, and, between said reflectors, a strip of material which is below said second path and which is not opaque to the radiation but which transmits the radiation upwardly from said emitter to said second path.

36. Apparatus according to claim 35, wherein said device further comprises first and second profiled strips also of material which is not opaque to the radiation, said strip and said profiled strips defining among them a channel shaped to guide said articles therealong.
